(19) United States
(12) Patent Application Publication
(10) Pub. No.: US 2025/0154142 A1
(43) Pub. Date: May 15, 2025

(54) THIADIAZOLE-SUBSTITUTED COMPOUND, PHARMACEUTICAL COMPOSITIONS THEREOF, AND APPLICATIONS THEREOF

(71) Applicant: Shanghai Yingli Pharmaceutical Co., Ltd., Shanghai (CN)

(72) Inventors: Zusheng XU, Shanghai (CN); Yangtong LOU, Shanghai (CN)

(73) Assignee: Shanghai Yingli Pharmaceutical Co., Ltd., Shanghai (CN)

(21) Appl. No.: 18/947,778

(22) Filed: Nov. 14, 2024

(30) Foreign Application Priority Data
Nov. 15, 2023 (CN) 2023115206769
Dec. 8, 2023 (CN) 2023116756889

(51) Int. Cl. C07D 419/14; A61K 31/496

(52) U.S. Cl. CPC C07D 419/14; A61K 31/496

(57) ABSTRACT

Disclosed is a thiadiazole-substituted compound, pharmaceutical composition thereof and application thereof. The present disclosure provides a compound containing structure of a thiadiazole-substituted compound represented by formula I, a pharmaceutically acceptable salt thereof, a stereoisomer thereof, an isomer thereof or an isotopic compound thereof. The thiadiazole-substituted compound has strong inhibitory effect on PARG and and better pharmacokinetics, is expected to treat and/or prevent various PARG-related diseases.

### CROSS-REFERENCE TO RELATED APPLICATIONS

This application claims priority under 35 U.S.C. § 119 (b) to Chinese Application No. 2023115206769, filed Nov. 15, 2023 and Chinese Application No. 2023116756889, filed Dec. 8, 2023, the disclosures of each of which are incorporated herein by reference in their entireties.

### TECHNICAL FIELD

The present disclosure relates to a thiadiazole-substituted compound, pharmaceutical compositions thereof, and applications thereof.

### BACKGROUND

Cancer cells tend to divide and proliferate uncontrollably, resulting in a higher incidence of DNA damage and defective DNA repair, and are more dependent on DNA damage repair mechanisms than normal cells.

Polyadenosine diphosphate ribose polymerase (PARP), polyadenosine diphosphate ribose hydrolase (PARG) and other proteins play an important role in DNA repair and have become important targets in the development of anti-cancer drugs. PARP can bind to single-strand break sites of DNA, which promotes the production of polyadenosine diphosphate ribose (PAR) chains, thus triggering the repair process. PARG is to degrade PAR on PARP and facilitate the completion of the entire repair cycle. Inhibition of either PARP or PARG affects the entire repair process.

Currently, the research and development of PARP inhibitors has been highly successful, with several drugs approved for marketing, and has demonstrated the feasibility of using DNA repair proteins as targets. At the same time, there is some problem still exist in PARP inhibitors such as not effective to all patients, drug resistance. The research on PARG inhibitors is still in the exploratory stage. There is a lot of room for research in this target direction, which is expected to fill the unmet clinical needs.

### Content of the Present Disclosure

The present disclosure provides a thiadiazole-substituted compound represented by formula I, a pharmaceutically acceptable salt thereof, a stereoisomer thereof, an isomer thereof or an isotopic compound thereof:

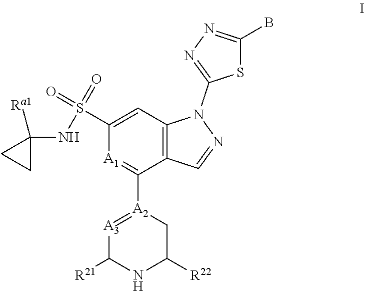

wherein, “ [symbol] ” represents a single bond or a double bond;
B is CF₃ or CHF₂;
A₁ is N or CR^{b1}, R^{b} is hydrogen or halogen; and when B is CHF₂, A₁ is CR^{b1}, R^{b1} is halogen;
A₂ is N, C or CR^{c1}; R^{c1} is hydrogen, halogen, C₁₋₆ alkyl or hydroxyl;
A₃ is CR²³ or CHR²³;
R^{a1} is cyano or C₁₋₆ alkyl;
R²¹ and R²² are independently hydrogen or C₁₋₆ alkyl, and when R²¹ is hydrogen, R²² is C₁₋₆ alkyl;
R²³ is hydrogen, halogen, hydroxyl, cyano, C₁₋₆ alkyl, C₁₋₆ alkyl substituted with one or more R²³⁻¹, C₂₋₆ alkenyl, C₂₋₆ alkynyl, —OC₁₋₆ alkyl, —SC₁₋₆ alkyl, —C(═O)R^{2a}, —NR^{2b1}R^{2b2}, —C(═O)OR^{2c}, —C(═O)NR^{2d1}R^{2d2}, C₃₋₁₀ cycloalkyl, “4- to 12-membered heterocycloalkyl containing 1 to 3 heteroatoms independently selected from O, S and N”, C₆₋₂₀ aryl, “5- to 12-membered heteroaryl containing 1 to 4 heteroatoms independently selected from O, S and N”, C₄₋₈ cycloalkenyl, or, “4- to 8-membered heterocycloalkenyl containing 1 to 3 heteroatoms independently selected from O, S and N”;
R²³⁻¹ is independently halogen, cyano, C₁₋₆ alkyl-O—, hydroxyl, —C(═O)R^{23a}, —NR^{23b1}R^{23b2}, C(═O)OR^{23c} or —C(═O)NR^{23d1}R^{23d2}, R^{2a}, R^{2b1}, R^{2b2}, R^{2c}, R^{2d1}, R^{2d2}, R^{23a}, R^{23b1}, R^{23b2}, R^{23c}, R^{23d1} and R^{23d2} are independently hydrogen, C₁₋₆ alkyl, or, C₁₋₆ alkyl substituted with one or more halogen;
or, R^{2b1} and R^{2b2}, R^{2d1} and R^{2d2}, R^{23b1} and R^{23b2}, R^{23d1} and R^{23d2} independently together with the N to which they are attached form “3- to 8-membered heterocycle containing 1 to 3 heteroatoms, wherein, one heteroatom is N, other heteroatoms, if any, independently selected from O, S and N”, or “3- to 8-membered heterocycle containing 1 to 3 heteroatoms, wherein, one heteroatom is N, other heteroatoms, if any, independently selected from O, S and N” substituted with one or more R¹⁻²⁻¹.

R¹⁻²⁻¹ is independently independently halogen, C₁₋₆ alkyl, or, C₁₋₆ alkyl substituted with one or more halogen.

In a certain embodiment, with regard to the thiadiazole-substituted compound represented by formula I, a pharmaceutically acceptable salt thereof, a stereoisomer thereof, an isomer thereof or an isotopic compound thereof, some groups are as defined as follows, and the unmentioned group definitions are as described in any one of the embodiments of the present disclosure (this content is hereinafter referred to simply as “in a certain embodiment”).

In a certain embodiment, in the thiadiazole-substituted compound represented by formula I, a pharmaceutically acceptable salt thereof, a stereoisomer thereof, an isomer thereof or an isotopic compound thereof, B is CF₃.

In a certain embodiment, in the thiadiazole-substituted compound represented by formula I,
wherein, “ [symbol] ” represents a single bond or a double bond;
B is CF₃ or CHF₂;
A₁ is N or CR^{b1}, R^{b1} is hydrogen or halogen, and when B is CHF₂, A₁ is CR^{b1}, R^{b1} is halogen;
A₂ is N or C;
A₃ is CR²³ or CHR²³;
R²³ is hydrogen;
R^{a1} is cyano or C₁₋₆ alkyl;
R²¹ and R²² are independently hydrogen or C₁₋₆ alkyl, and when R²¹ is hydrogen, R²² is C₁₋₆ alkyl.

In a certain embodiment, the thiadiazole-substituted compound represented by formula I, having the structure represented by formula II:

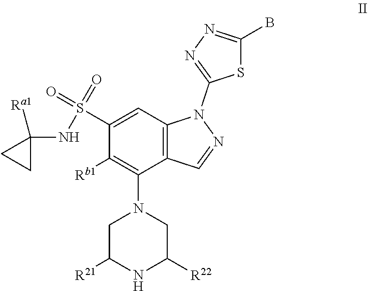

wherein, B is CF₃ or CHF₂;
R^{b1} is hydrogen or halogen, and when B is CHF₂, R^{b1} is halogen;
R^{a1} is cyano or C₁₋₆ alkyl;
R²¹ and R²² are independently C₁₋₆ alkyl.

In a certain embodiment, A₂ is N or C.

In a certain embodiment, A₃ is CR²³ or CHR²³; R²³ is hydrogen.

In a certain embodiment, “ [symbol] ” is a single bond, A₂ is N, A₃ is CH₂, R²¹ and R²² are independently C₁₋₆ alkyl.

In a certain embodiment, when R²¹ and R²² are independently C₁₋₆ alkyl,

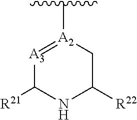

is

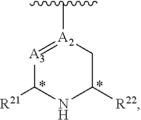

wherein,

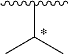 [SMILES: *C(C)C]

represents R conformation, S conformation or a mixture of R and S conformation.

In a certain embodiment, when the definition of R^{b1}, R^{c1}, R²³, R²³⁻¹, R^{2a}, R^{2b1}, R^{2b2}, R^{2c}, R^{2d1}, R^{2d2}, R^{23a}, R^{23b1}, R^{23b2}, R^{23c}, R^{23d1}, R^{23d2} and R¹⁻²⁻¹ refers to halogen, the halogen is fluorine, chlorine, bromine or iodine.

In a certain embodiment, when the definition of R^{c1}, R^{a1}, R²¹, R²², R²³, R^{2a}, R^{2b1}, R^{2b2}, R^{2c}, R^{2d1}, R^{2d2}, R^{23a}, R^{23b1}, R^{23b2}, R^{23c}, R^{23d1}, R^{23d2} and R¹⁻²⁻¹ refers to C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment, A₁ is CF or CH.

In a certain embodiment, R^{a1} is cyano or methyl.

In a certain embodiment, R²¹ and R²² are independently hydrogen or methyl, and when R²¹ is hydrogen, R²² is methyl.

In a certain embodiment,

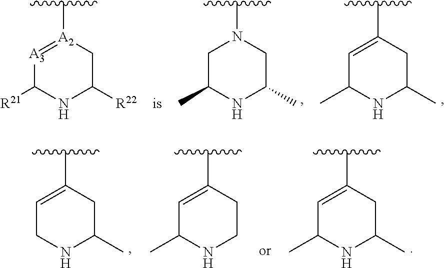

In a certain embodiment, in the thiadiazole-substituted compound represented by formula I, a pharmaceutically acceptable salt thereof, a stereoisomer thereof, an isomer thereof or an isotopic compound thereof, the thiadiazole-substituted compound represented by formula I is any one of the following structures:

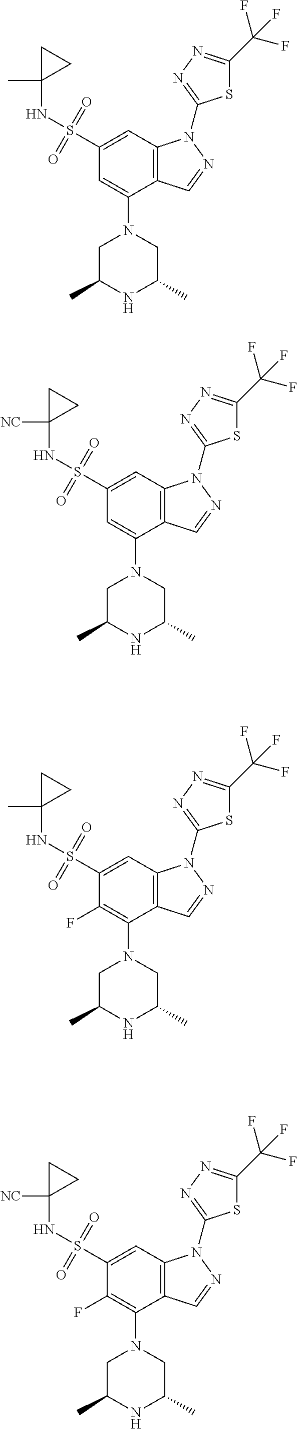 [SMILES: C[C@H]1CN(c2cc(S(=O)(=O)NC3(C)CC3)cc3c2cnn3-c2nnc(C(F)(F)F)s2)C[C@H](C)N1 ; C[C@H]1CN(c2cc(S(=O)(=O)NC3(C#N)CC3)cc3c2cnn3-c2nnc(C(F)(F)F)s2)C[C@H](C)N1 ; C[C@H]1CN(c2c(F)c(S(=O)(=O)NC3(C)CC3)cc3c2cnn3-c2nnc(C(F)(F)F)s2)C[C@H](C)N1 ; C[C@H]1CN(c2c(F)c(S(=O)(=O)NC3(C#N)CC3)cc3c2cnn3-c2nnc(C(F)(F)F)s2)C[C@H](C)N1]

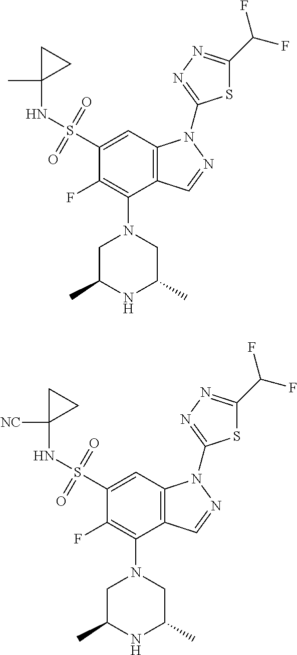 [SMILES: C[C@H]1CN(c2c(F)c(S(=O)(=O)NC3(C)CC3)cc3c2cnn3-c2nnc(C(F)F)s2)C[C@H](C)N1 ; C[C@H]1CN(c2c(F)c(S(=O)(=O)NC3(C#N)CC3)cc3c2cnn3-c2nnc(C(F)F)s2)C[C@H](C)N1]

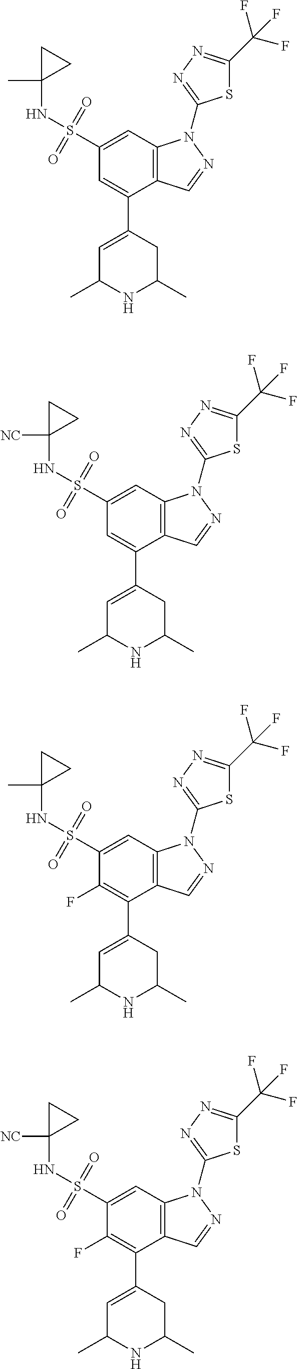 [SMILES: CC1C=C(c2cc(S(=O)(=O)NC3(C)CC3)cc3c2cnn3-c2nnc(C(F)(F)F)s2)CC(C)N1 ; CC1C=C(c2cc(S(=O)(=O)NC3(C#N)CC3)cc3c2cnn3-c2nnc(C(F)(F)F)s2)CC(C)N1 ; CC1C=C(c2c(F)c(S(=O)(=O)NC3(C)CC3)cc3c2cnn3-c2nnc(C(F)(F)F)s2)CC(C)N1 ; CC1C=C(c2c(F)c(S(=O)(=O)NC3(C#N)CC3)cc3c2cnn3-c2nnc(C(F)(F)F)s2)CC(C)N1]

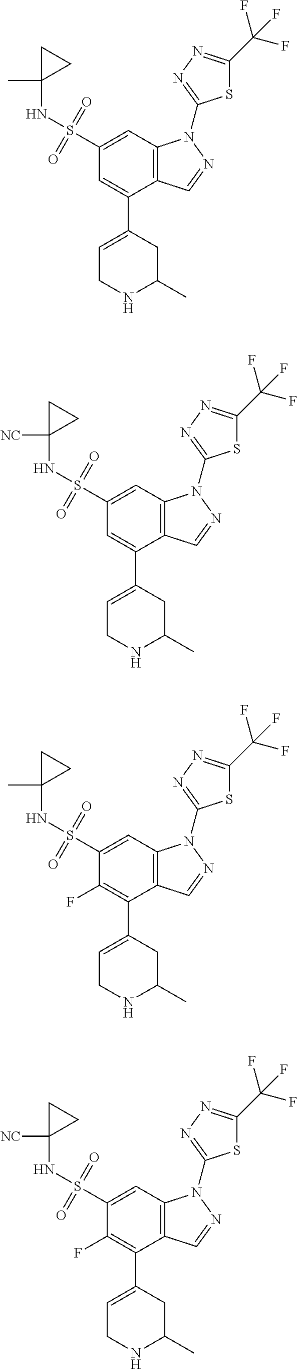 [SMILES: CC1CC(c2cc(S(=O)(=O)NC3(C)CC3)cc3c2cnn3-c2nnc(C(F)(F)F)s2)=CCN1 ; CC1CC(c2cc(S(=O)(=O)NC3(C#N)CC3)cc3c2cnn3-c2nnc(C(F)(F)F)s2)=CCN1 ; CC1CC(c2c(F)c(S(=O)(=O)NC3(C)CC3)cc3c2cnn3-c2nnc(C(F)(F)F)s2)=CCN1 ; CC1CC(c2c(F)c(S(=O)(=O)NC3(C#N)CC3)cc3c2cnn3-c2nnc(C(F)(F)F)s2)=CCN1]

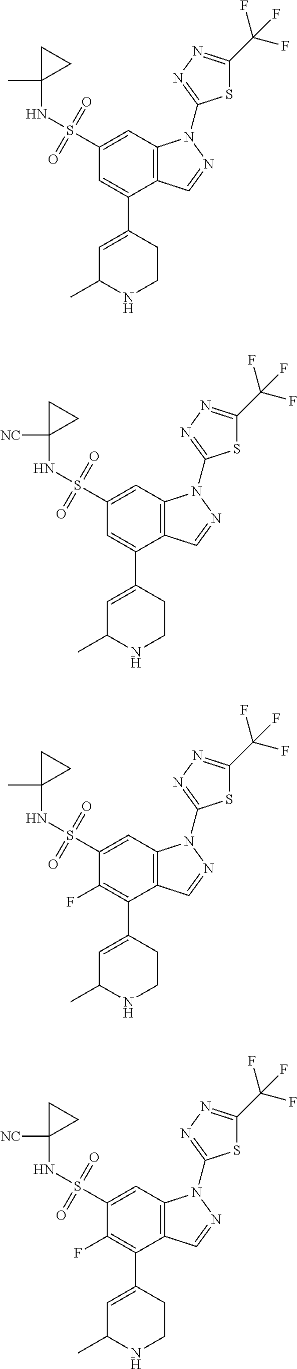 [SMILES: CC1C=C(c2cc(S(=O)(=O)NC3(C)CC3)cc3c2cnn3-c2nnc(C(F)(F)F)s2)CCN1 ; CC1C=C(c2cc(S(=O)(=O)NC3(C#N)CC3)cc3c2cnn3-c2nnc(C(F)(F)F)s2)CCN1 ; CC1C=C(c2c(F)c(S(=O)(=O)NC3(C)CC3)cc3c2cnn3-c2nnc(C(F)(F)F)s2)CCN1 ; CC1C=C(c2c(F)c(S(=O)(=O)NC3(C#N)CC3)cc3c2cnn3-c2nnc(C(F)(F)F)s2)CCN1]

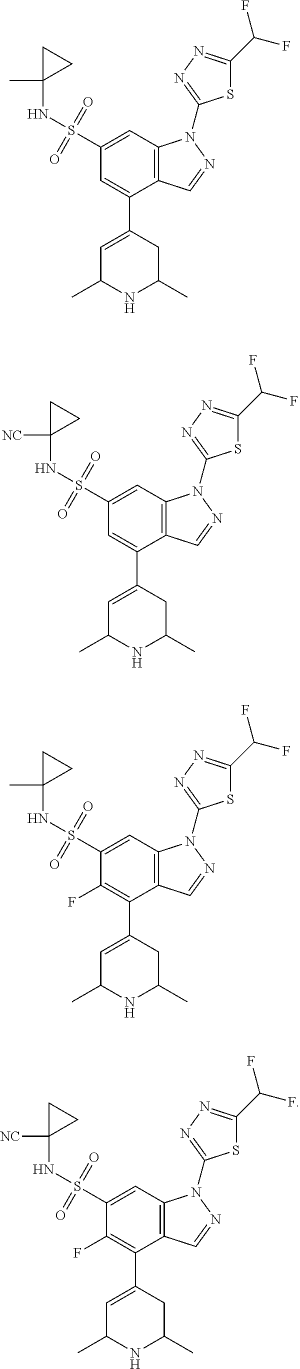 [SMILES: CC1C=C(c2cc(S(=O)(=O)NC3(C)CC3)cc3c2cnn3-c2nnc(C(F)F)s2)CC(C)N1 ; CC1C=C(c2cc(S(=O)(=O)NC3(C#N)CC3)cc3c2cnn3-c2nnc(C(F)F)s2)CC(C)N1 ; CC1C=C(c2c(F)c(S(=O)(=O)NC3(C)CC3)cc3c2cnn3-c2nnc(C(F)F)s2)CC(C)N1 ; CC1C=C(c2c(F)c(S(=O)(=O)NC3(C#N)CC3)cc3c2cnn3-c2nnc(C(F)F)s2)CC(C)N1]

The present disclosure also provides a pharmaceutical composition comprising a substance A and a pharmaceutically acceptable excipient, wherein the substance A is a therapeutically effective amount of the thiadiazole-substituted compound represented by formula I, the pharmaceutically acceptable salt thereof, the stereoisomer thereof, the tautomer thereof or the isotopically labeled compound thereof as described above.

The present disclosure also provides a method of inhibiting PARG in a subject in need thereof, comprising: administering a therapeutically effective amount of a substance A to the subject, wherein the substance A is the thiadiazole-substituted compound represented by formula I, the pharmaceutically acceptable salt thereof, the stereoisomer thereof, the tautomer thereof or the isotopically labeled compound thereof as described above.

The present disclosure also provides a method of treating or preventing a PARG related disease in a subject in need thereof, comprising: administering an effective amount of a substance A, wherein the substance A is the thiadiazole-substituted compound represented by formula I, the pharmaceutically acceptable salt thereof, the stereoisomer thereof, the tautomer thereof or the isotopically labeled compound thereof as described above.

In the method for treating or preventing an PARG related disease in a subject in need thereof, wherein the PARG related disease is cancer, the cancer is selected from the group consisting of colon cancer, appendicle cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The present disclosure also provides a method for treating or preventing a cancer, comprising: administering a therapeutically effective amount of a substance A to the subject, wherein the substance A is the thiadiazole-substituted compound represented by formula I, the pharmaceutically acceptable salt thereof, the stereoisomer thereof, the tautomer thereof or the isotopically labeled compound thereof as described above; the cancer is selected from the group consisting of colon cancer, appendicle cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The term “pharmaceutically acceptable salt” refers to a salt prepared from compounds of the present disclosure with relatively non-toxic, pharmaceutically acceptable acids or bases. When compounds of the present disclosure contain relatively acidic functional groups, base addition salts can be obtained by contacting the neutral form of such compounds with a sufficient amount of pharmaceutically acceptable bases, either in pure solution or a suitable inert solvent. The pharmaceutically acceptable base addition salts include but are not limited to: lithium salt, sodium salt, potassium salt, calcium salt, aluminum salt, magnesium salt, zinc salt, bismuth salt, ammonium salt and diethanolamine salt. When compounds of the present disclosure contain relatively basic functional groups, acid addition salts can be obtained by contacting the neutral form of such compounds with a sufficient amount of pharmaceutically acceptable acids, either in pure solution or a suitable inert solvent. The pharmaceutically acceptable acids include inorganic acids, and the inorganic acids include but are not limited to: hydrochloric acid, hydrobromic acid, hydroiodic acid, nitric acid, carbonic acid, phosphoric acid, phosphorous acid and sulfuric acid. The pharmaceutically acceptable acids include organic acids, and the organic acids include but are not limited to: acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, hydrogen tartrate, ascorbic acid, gentisic acid, fumaric acid, gluconic acid, saccharic acid, formic acid, ethanesulfonic acid, pamoic acid (i.e., 4,4′-methylene-bis(3-hydroxy-2-naphthoic acid)) and amino acid (such as glutamic acid and arginine). When compounds of the present disclosure contain relatively acidic functional groups and relatively basic functional groups, such compounds can be converted into base addition salts or acid addition salts. For details, reference can be made to Berge et al., “Pharmaceutical Salts”, Journal of Pharmaceutical Science 66:1-19 (1977), or Handbook of Pharmaceutical Salts: Properties, Selection, and Use (P. Heinrich Stahl and Camille G. Wermuth, ed., Wiley-VCH, 2002).

In the present disclosure, when multiple substituents are present, the substituents are the same or different.

The term “stereoisomer” refers to an isomer in which the atoms or atomic groups in a molecule have the same interconnection order but different spatial arrangements, such as cis-trans isomers, optical isomers or atropisomers. These stereoisomers can be separated, purified and enriched by means of asymmetric synthesis methods or chiral separation methods (including but not limited to thin layer chromatography, rotation chromatography, column chromatography, gas chromatography and high-pressure liquid chromatography) or can also be obtained by means of chiral resolution via forming bonds (chemical bonding, etc.) or forming salts (physical bonding) with other chiral compounds, etc.

The term “tautomer” refers to a functional group isomer resulting from the rapid movement of an atom in two positions in a molecule. For example, acetone and 1-propene-2-ol can be converted into each other by the rapid movement of hydrogen atoms on oxygen and α-carbon.

The term “hydroxyl” refers to a —OH group.

The term “cyano” refers to a —CN group.

The terms “cycloalkyl” and “carbocyclic ring” refer to a saturated cyclic group consisting only of carbon atoms having a specified number of carbon atoms (e.g., C₃-C₆), which is a monocyclic, bridged or spiro ring. The cycloalkyl includes, but is not limited to, cyclopropyl, cyclobutyl, cyclopentyl, and cyclohexyl.

The term “aryl” refers to an aromatic group consisting of carbon atoms, each ring having aromaticity. For example, phenyl or naphthyl.

The term “heteroaryl” refers to a cyclic group having a specified number of ring atoms (e.g., 5-12 members), a specified number of heteroatoms (e.g., 1, 2, or 3) and specified heteroatom species (one or more of N, O and S), which is monocyclic or polycyclic, and has at least one aromatic ring (according to the Hückel's rule). Heteroaryls are linked to other fragments of the molecule through aromatic or non-aromatic rings. Heteroaryls include, but are not limited to, furyl, pyrrolyl, thienyl, pyrazolyl, imidazolyl, oxazolyl, thiazolyl, pyridinyl, pyrimidinyl, and indolyl.

The term “isotopic compound” refers to a compound in which one or more atoms are substituted with one or more atoms having a specific atomic mass or mass number. Examples of isotopes that can be incorporated into compounds of the present disclosure include, but are not limited to, isotopes of hydrogen, carbon, nitrogen, oxygen, fluorine, sulfur and chlorine (e.g., ²H, ³H, ¹³C, ¹⁴C, ¹⁵N, ¹⁸O, ¹⁷O, ¹⁸F, ³⁵S and ³⁶Cl). The isotopic compounds of the present disclosure can generally be prepared by substituting non-isotopically-labeled reagents with isotopically-labeled reagents according to the methods described herein.

The term “halogen” refers to fluorine, chlorine, bromine or iodine.

The term “alkyl” refers to a linear or branched alkyl group having a specified number of carbon atoms. Examples of alkyl include methyl, ethyl, n-propyl, isopropyl, n-butyl, tert-butyl, isobutyl, sec-butyl, n-pentyl, n-hexyl, n-heptyl, n-octyl, and the like.

The above preferred conditions may be combined arbitrarily to obtain preferred embodiments of the present disclosure without departing from the general knowledge in the art.

The reagents and starting materials used in the present disclosure are commercially available.

The positive effects of the present disclosure are as follows: the present disclosure provides a thiadiazole-substituted compound, pharmaceutical compositions thereof and applications thereof, and the thiadiazole-substituted compound represented by formula I is expected to treat and/or prevent various PARG-related diseases.

### DETAILED DESCRIPTION OF THE EMBODIMENTS

The present disclosure is further illustrated by the following examples, which are not intended to limit the present disclosure. Experimental procedures without specified conditions in the following examples were performed in accordance with conventional procedures and conditions, or in accordance with instructions. The solvents involved in the following embodiments are analytically or chromatographically pure. When the solvents involved in the following embodiments are mixed solvents, they are all volume ratios unless otherwise stated.

The following is a list of abbreviations used in the embodiments:
DCM dichloromethane
DMF N, N-dimethylformamide
DMSO Dimethyl sulfoxide
DIPEA diisopropylethylamine
DBU 1,8-Diazabicyclo[5.4.0]undec-7-ene
NMP N-methylpyrrolidone
Pd₂(dba)₃ tris(dibenzylideneacetone) dipalladium
Xantphos 4,5-Bis(diphenylphosphino)-9,9-dimethylxanthene
SelectFluor II I-Fluoro-4-methyl-1,4-diazabicyclo[2.2.2]octane-1,4-diium tetrafluoroborate

### Example 1 Synthetic Route of Compound 1

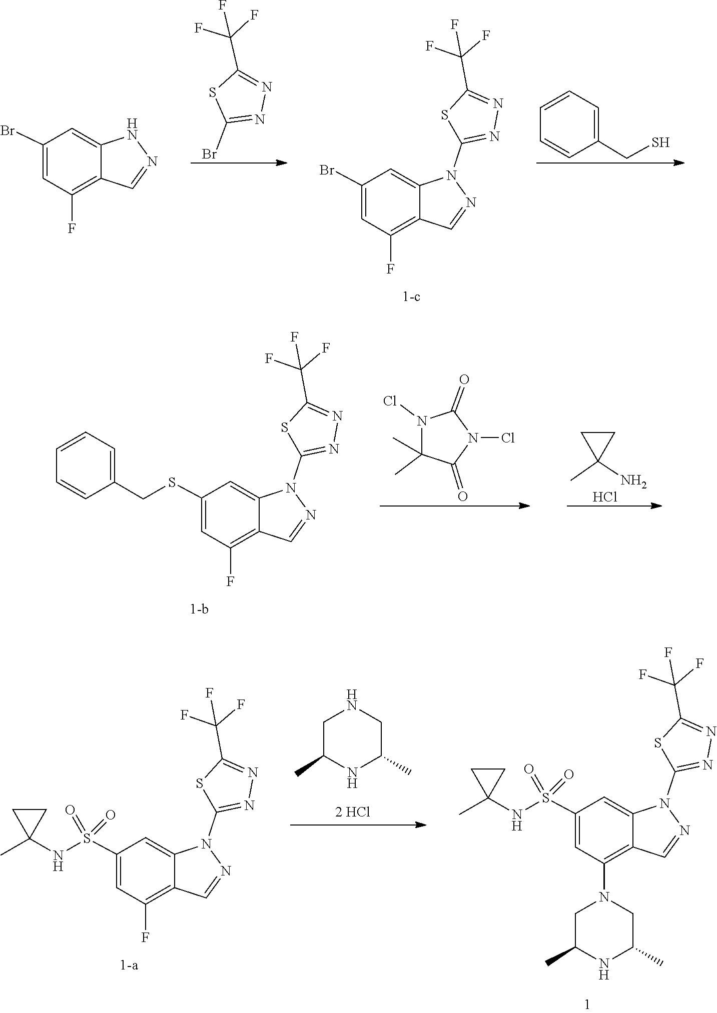

### Synthesis of Compounds 1-c

2-Bromo-5-(trifluoromethyl)-1,3,4-thiadiazole (678 mg, 2.91 mmol) and anhydrous DMF (8 mL) were added into a microwave tube. 6-Bromo-4-fluoro-1H-indazole (500 mg, 2.33 mmol) and cesium carbonate (1820 mg, 5.59 mmol) were added at room temperature and sealed. The reaction mixture was heated and stirred at 60° C. for 2 hours. The reaction mixture was cooled to room temperature, added into ice water, acidified by adding solid sodium bisulfate and extracted with ethyl acetate twice. The organic phases were combined, washed with brine, dried over anhydrous sodium sulfate, filtered, concentrated to dryness by rotary evaporation, and dried by an oil pump to give 1-c (852 mg, 100%).

### Synthesis of Compound 1-b

In a reaction vial 1-c (750 mg, 2.04 mmol), Pd₂(dba)₃ (90 mg, 0.098 mmol), XANT PHOS (90 mg, 0.16 mmol), DIPEA (792 mg, 6.13 mmol), 1,4-dioxane (20 mL), and benzyl mercaptan (0.31 mL, 2.66 mmol). After degassed and purged with nitrogen for three times, the reaction mixture was heated and stirred at 90° C. for 1.5 hours, monitored, it was not finished, then stirred at 90° C. for another 1.5 hours. Cooled to room temperature, the reaction mixture was removed solvent by rotary evaporation, and the residue was added ethyl acetate and petroleum ether, washed with water, brine, dried over anhydrous sodium sulfate, filtered, evaporated to dryness, and purified by column chromatography (mobile phase: petroleum ether/ethyl acetate, 100/0 to 90/10) to obtain compound 1-b (800 mg, 95% yield). LC-MS (ESI): m/z 411.1 (M+H)⁺.

### Synthesis of Compound 1-a

To a reaction flask were added 1-b (800 mg, 1.95 mmol), acetonitrile (20 mL), acetic acid (0.13 mL, 2.33 mmol) and water (0.14 mL, 7.77 mmol). Dichlorhydantoin (768 mg, 3.90 mmol) was added to the above mixture in an ice-water bath. The reaction mixture was stirred in an ice-water bath for 1 h. The solvent was removed by rotary evaporation at room temperature. The residue was added ethyl acetate and petroleum ether, washed with water, brine, dried over anhydrous sodium sulfate, filtered, evaporated to dryness, and dried by an oil pump to obtain the crude product. The crude product was dissolved in anhydrous dichloromethane (10 mL) to obtain solution A. To a reaction flask were added 1-methylcyclopropanamine hydrochloride (420 mg, 3.90 mmol), triethylamine (1.36 mL, 9.75 mmol) and anhydrous dichloromethane (7 mL) in an ice-water bath. The reaction mixture was stirred for 5 min in an ice-water bath and then was added Solution A, and anhydrous dichloromethane (4 mL) washed the flask that had contained the Solution A twice and added into the above reaction mixture. The reaction mixture was stirred at room temperature for 1 h, then was washed with water, brine, dried over anhydrous sodium sulfate, filtered, evaporated, and purified by column chromatography (mobile phase: petroleum ether/ethyl acetate, 100/0 to 80/20) to give compound 1-a (330 mg, 40% yield). LC-MS (ESI): m/z 422.0 (M+H)⁺.

### Synthesis of Compound 1

1-a (90 mg, 0.21 mmol) and (2S,6S)-2,6-dimethylpiperazine dihydrochloride (120 mg, 0.64 mmol) were added to a reaction flask. The mixture was degassed to vacuum and added anhydrous NMP (4 mL). After degassed and purged with nitrogen for 3 times, the above mixture was added DBU (195 mg, 1.28 mmol). The reaction mixture was heated and stirred at 80° C. for 6 hours. The reaction mixture was cooled to room temperature, added water, extracted with ethyl acetate twice, dried over anhydrous sodium sulfate, filtered, evaporated, and purified by column chromatography (C8 column, mobile phase: 10 mM ammonium bicarbonate, acetonitrile) to obtain the crude (95% purity), and then purified on a silica column again (mobile phase: dichloromethane/methanol, 100/0 to 94/6) to obtain compound 1 (61 mg, 55% yield). LC-MS (ESI): m/z 516.2 (M+H)⁺; ¹H NMR (DMSO-d₆, 400 MHZ) δ 8.80 (1H, s), 8.39 (1H, s), 8.34 (1H, s), 7.13 (1H, s), 3.43-3.37 (4H, m), 3.11 (2H, dd, J=12.0, 6.4 Hz), 1.19 (6H, d, J=6.4 Hz), 1.08 (3H, s), 0.71-0.61 (2H, m), 0.45-0.36 (2H, m).

### Example 2 Synthetic Route of Compound 2

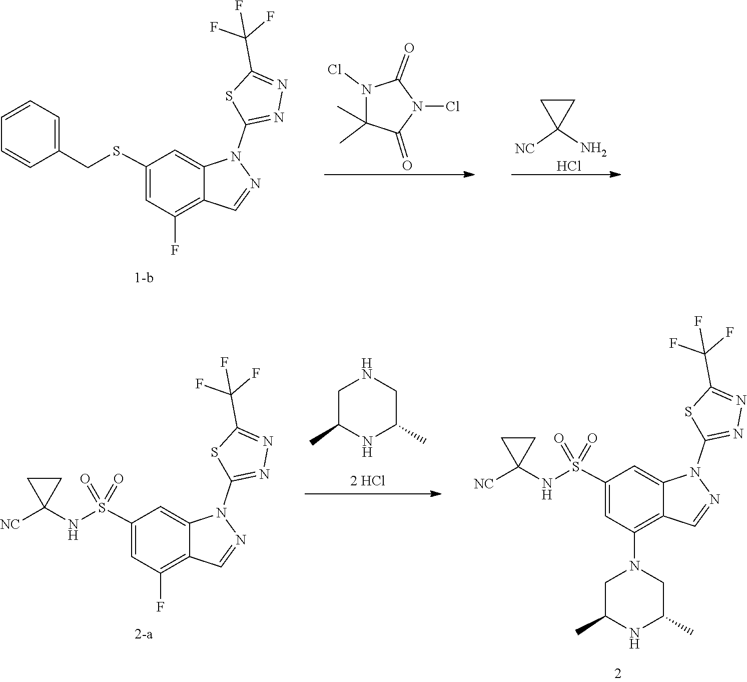

### Synthesis of Compound 2-a

To a reaction flask were added 1-b (270 mg, 0.66 mmol), acetonitrile (11 mL), acetic acid (0.043 mL, 0.75 mmol) and water (0.045 mL, 2.50 mmol). Dichlorhydantoin (266 mg, 1.35 mmol) was added to the above mixture in an ice-water bath. The reaction mixture was stirred in an ice-water bath for 1 hour and concentrated to dryness by rotary evaporation at low temperature.

The residue was added ethyl acetate and petroleum ether, washed with water, dried over anhydrous sodium sulfate, filtered, evaporated to dryness, and dried by an oil pump for 10 min. added dichloromethane (5 mL) to give Solution A. To a reaction flask was added 1-amino-1-cyclopropyl cyanide hydrochloride (312 mg, 2.63 mmol), and to this flask were added pyridine (0.5 mL), dichloromethane (6 mL) and the Solution A sequentially under an ice-water bath. The reaction mixture was stirred at room temperature for 3 h. Sodium bisulfate aqueous solution was added. Aqueous sodium bisulfate was added, partitioned, and the organic phase was dried over anhydrous sodium sulfate, filtered, and evaporated to dryness. The crude was purified by column chromatography (mobile phase: dichloromethane/methanol, 100/0 to 95/5) to afford compound 2-a (170 mg, 60% yield). LC-MS (ESI): m/z 433.1 (M+H)⁺.

### Synthesis of Compound 2

2-a (70 mg, 0.162 mmol) and (2S,6S)-2,6-dimethylpiperazine dihydrochloride (90.6 mg, 0.484 mmol) were added to a reaction vial. The above mixture was degassed to vacuum and was added anhydrous NMP (2 mL). Degassed and purged with nitrogen twice and DBU (138 mg, 0.906 mmol) was added. The reaction mixture was heated and stirred at 80° C. for 6 hours, then was cooled to room temperature, added water and extracted twice with ethyl acetate. The organic phases were combined, dried over anhydrous sodium sulfate, filtered, evaporated, and the residue was purified by column chromatography (mobile phase: dichloromethane/(dichloromethane/methanol/ammonia 90:10:0.2), 100/0 to 35/65), and lyophilized in acetonitrile diluted with ammonia to give compound 2 (51 mg, 60% yield). LC-MS (ESI): m/z 527.1 (M+H)⁺. ¹H NMR (DMSO-d₆, 400 MHz) δ 8.84 (1H, s), 8.42 (1H, s), 7.14 (1H, s), 3.42 (2H, dd, J=11.6, 2.4 Hz), 3.37-3.34 (2H, m), 3.12 (2H, dd, J=11.6, 6.4 Hz), 1.47-1.39 (2H, m), 1.36-1.26 (2H, m), 1.19 (6H, d, J=6.4 Hz).

### Example 3 Synthetic Route of Compound 3

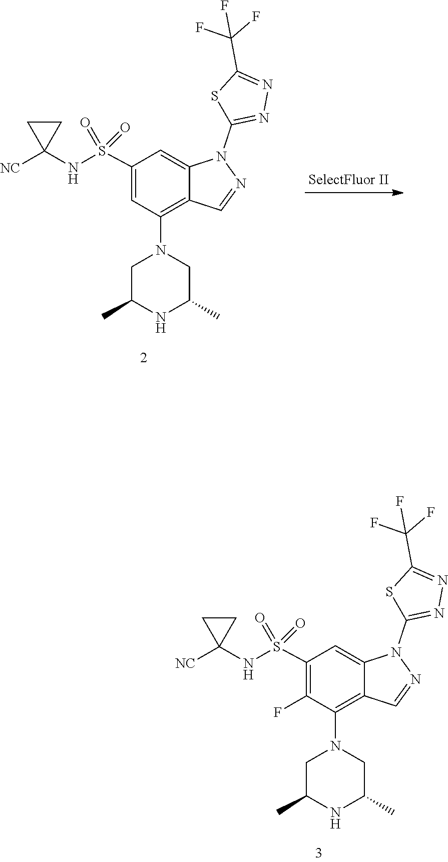 [SMILES: C[C@H]1CN(c2cc(S(=O)(=O)NC3(C#N)CC3)cc3c2cnn3-c2nnc(C(F)(F)F)s2)C[C@H](C)N1 ; C[C@H]1CN(c2c(F)c(S(=O)(=O)NC3(C#N)CC3)cc3c2cnn3-c2nnc(C(F)(F)F)s2)C[C@H](C)N1]

### Synthesis of Compound 3

Compound 2 (50 mg, 0.095 mmol) and SelectFluor II (91 mg, 0.29 mmol) were combined in a reaction flask. After degassed to vacuum, the above mixture was added anhydrous acetonitrile (5 mL). Degassed and purged with nitrogen for 2 times, the above mixture was added acetic acid (0.027 mL, 0.47 mmol). The reaction mixture was stirred at 40° C. for 4.5 hours, then was cooled to room temperature, added ethyl acetate, pure water and anhydrous sodium sulfate, partitioned, and the aqueous phase was extracted with ethyl acetate once. The organic phases were combined, dried over anhydrous sodium sulfate, filtered, evaporated and purified by prep-HPLC (ammonium bicarbonate) to give compound 3 (11.2 mg, 22% yield). LC-MS (ESI): m/z 545.7 (M+H)⁺; ¹H NMR (DMSO-d₆, 400 MHz) δ 8.94 (1H, s), 8.51 (1H, d, J=4.4 Hz), 3.53-3.49 (4H, m), 3.17 (2H, dd, J=12.0, 6.0 Hz), 1.46-1.37 (2H, m), 1.34-1.27 (2H, m), 1.20 (6H, d, J=6.4 Hz).

### Example 4 Synthetic Route of Compound 4

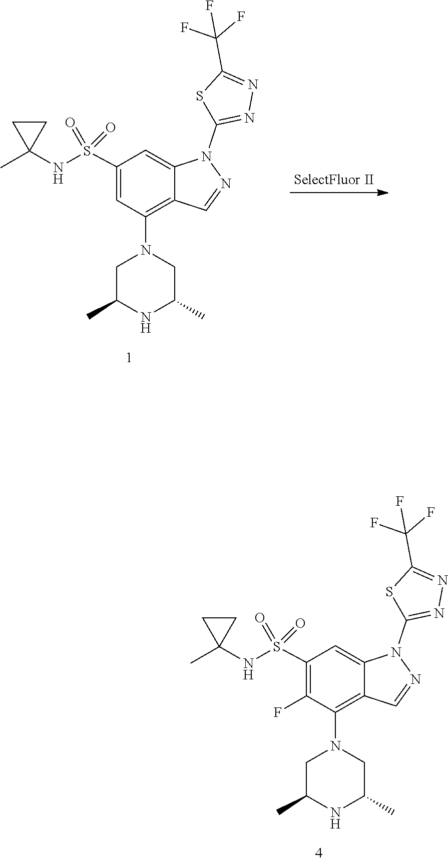 [SMILES: C[C@H]1CN(c2cc(S(=O)(=O)NC3(C)CC3)cc3c2cnn3-c2nnc(C(F)(F)F)s2)C[C@H](C)N1 ; C[C@H]1CN(c2c(F)c(S(=O)(=O)NC3(C)CC3)cc3c2cnn3-c2nnc(C(F)(F)F)s2)C[C@H](C)N1]

### Synthesis of Compound 4

Compound 1 (70 mg, 0.14 mmol) and SelectFluor II (130 mg, 0.41 mmol) were added to a reaction flask. After degassed to vacuum, the mixture was added anhydrous acetonitrile (5 mL), then degassed and purged with nitrogen for 2 times, the mixture was added acetic acid (0.029 mL, 0.50 mmol). The reaction mixture was stirred at room temperature for 10 hours, then was added ethyl acetate, pure water and anhydrous sodium sulfate, partitioned and the aqueous phase was extracted with ethyl acetate once. The organic phases were combined, dried over anhydrous sodium sulfate, filtered, evaporated and purified by prep-HPLC (ammonium bicarbonate) to give compound 4 (19.2 mg, 28% yield). LC-MS (ESI): m/z 534.3 (M+H)⁺. ¹H NMR (DMSO-d₆, 400 MHz) δ 8.91 (1H, d, J=0.4 Hz), 8.59 (1H, brs), 8.45 (1H, d, J=4.8 Hz), 3.50-3.41 (2H, m), 3.31-3.22 (2H, m), 3.10 (2H, dd, J=11.2, 6.0 Hz), 1.17-1.12 (9H, m), 0.74-0.65 (2H, m), 0.47-0.41 (2H, m).

### Synthesis of Comparative Compound 1′

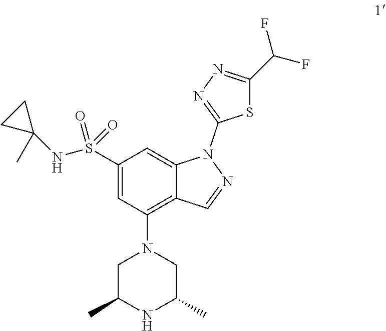 [SMILES: C[C@H]1CN(c2cc(S(=O)(=O)NC3(C)CC3)cc3c2cnn3-c2nnc(C(F)F)s2)C[C@H](C)N1]

Synthesized according to the synthetic method of patent WO2023183850A1.

### Example 5 Synthetic Route of Compound 5

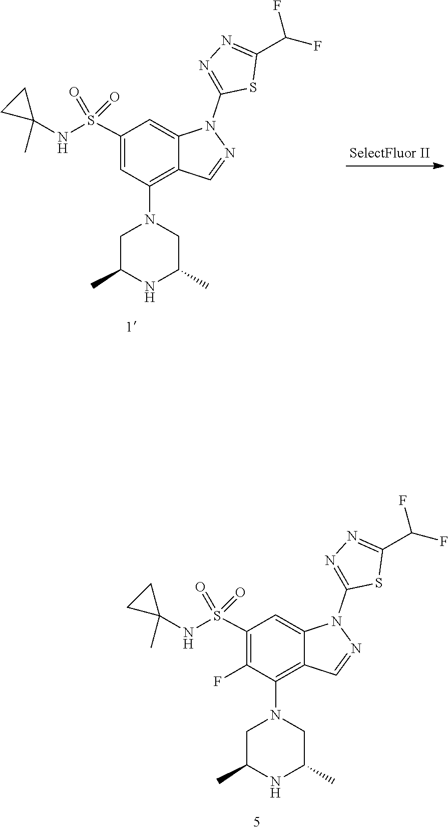 [SMILES: C[C@H]1CN(c2cc(S(=O)(=O)NC3(C)CC3)cc3c2cnn3-c2nnc(C(F)F)s2)C[C@H](C)N1 ; C[C@H]1CN(c2c(F)c(S(=O)(=O)NC3(C)CC3)cc3c2cnn3-c2nnc(C(F)F)s2)C[C@H](C)N1]

### Synthesis of Compound 5

Compound 1′ (160 mg, 0.32 mmol), 1-fluoro-4-methyl-1,4-diazabicyclo[2.2.2]octane tetrafluoroborate (309 mg, 0.97 mmol), and acetic acid (525 mg, 8.74 mmol) were added to acetonitrile (10 mL), and the reaction mixture was stirred for 10 hr at room temperature after the reaction mixture was degassed and purged with nitrogen for three times. The pH was adjusted to 7˜8 by adding saturated sodium bicarbonate solution to the reaction mixture, to which water (50 mL) was added and extracted with ethyl acetate (50 mL). The organic phase was washed with brine (50 mL), dried over sodium sulfate, filtered off the desiccant, and the filtrate was concentrated under reduced pressure to obtain the crude product, which was purified by column chromatography (mobile phase, DCM/MeOH 20/1), and then purified by a C18 column (NH₄HCO₃) to obtain compound 5 (24 mg, 14%). LC-MS (ESI): m/z 516.4 (M+H)⁺; ¹H NMR (400 MHz, DMSO-d₆): δ 8.89 (1H, s), 8.60 (1H, s), 8.49 (1H, d, J=4.0 Hz), 7.61 (1H, t, J=52.0 Hz), 3.55-3.46 (2H, m), 3.43-3.36 (2H, m), 3.20-3.11 (2H, m), 1.25-1.18 (7H, m), 1.14 (3H, s), 0.75-0.67 (2H, m), 0.50-0.40 (2H, m).

### Effectiveness Data

### Bioactivity Test

### PARG Enzyme Inhibition Assay

### Experimental Procedure.

PARG in vitro assays were conducted in standard 384-well plates in a total volume of 15 μL. 5 μL of PARG (389-976) (manufactured by Chempartner Chemical Co., Ltd.) in buffer (50 mM Tris-HCL 7.5, 30 mM KCl, 1 mM EDTA, 3 mM DTT, tween-20 0.01%, BSA 0.025%) was added at a final concentration of 1.5 pM to the 384-well plates containing the compounds to be tested, which was incubated for 30 min at room temperature. To the above mixture was added 5 μL Bio PARylated His-TEV-PARP1 (2-1014) substrate (manufactured by Chempartner Chemical Co., Ltd.) at a final concentration of 12 nM, after addition, the resulting mixture was incubated for 30 minutes at room temperature. Then to the mixture was added detection reagent (5 μL) which was buffered with 50 mM Tris-HCL 7.5, 30 mM KCl, 1 mM EDTA, 3 mM DTT, tween-20 0.01%, BSA 0.025%, and consisted of 3 μM of compound PDD00017273 and 9 nM Mab anti-6HIS XL665 (Cisbio: 61HISXLA) and 0.9 nM streptavidin affinity terbium cryptate (Cisbio: 610SATLA), all at 3× working concentrations (final concentrations of 1 M, 3 nM and 0.3 nM, respectively). After 120 min incubation in the dark at room temperature, TR-FRET signals were measured at Ex 340 and Em 665 and Em 615. The ratio for each well was calculated as Em 665/Em 615 and the compound inhibition rate was calculated based on the obtained data.

### PARG Biochemical Activity of Representative Compounds of the Present Disclosure

| | IC₅₀ | | IC₅₀ | | IC₅₀ |
|---|---|---|---|---|---|
| Compound | (PARG | Compound | (PARG | Compound | (PARG |
| No. | enzyme) | No. | enzyme) | No. | enzyme) |
| 1 | ***** | 2 | ***** | 1′ | ***** |
| | (0.20 nM) | | (0.62 nM) | | (0.21 nM) |
| 4 | ***** | 5 | ***** | | |

***** represents IC₅₀ < 2 nM;
**** represents 2 nM ≤ IC₅₀ < 10 nM;
*** represents 10 nM ≤ IC₅₀ < 100 nM;
** represents 100 nM ≤ IC₅₀ < 1 μM;
* represents IC₅₀ ≥ 1 μM

Pharmacokinetic Studies in Mice after Intravenous and Oral Administration of the Drug Experimental Procedure:

After intravenous and oral administration to male C57BL/6J mice or male CD-1 mice, plasma samples were collected through the orbital venous plexus at 8-9 time points (e.g., 5 min, 15 min, 30 min, 1 h, 2 h, 4 h, 7 h, 24 h or 5 min, 15 min, 30 min, 1 h, 2 h, 4 h, 6 h, 8 h, 24 h). The collected blood samples were transferred to microcentrifuge tubes containing EDTA-K2 anticoagulant, and the supernatants were centrifuged for 5 min at 4° C. and 4000 g. The supernatants were stored in a refrigerator at −75° C.±15° C. The concentrations of the compounds in the plasma at the different time points were detected by LC-MS/MS and the relevant pharmacokinetic parameters were calculated using the WinNonlin software

### Results of Pharmacokinetic Testing of Representative Compounds of the Present Disclosure

| | | Test compounds | | |
|---|---|---|---|---|
| | | compound | Compound | Compound |
| way of administration | | 1^{b} | 1′^{b} | 3^{b} |
| intravenous^{a1} | Cl | 22.5 | 89.5 | 34.8 |
| (5 mg/kg) | (mL/min/kg) | | | |
| | AUC₀₋ₗₐₛₜ | 2539 | 905 | 2407 |
| | (h*ng/mL) | | | |
| | AUC_{0-inf} | 3703 | 939 | 2417 |
| | (h*ng/mL) | | | |
| oral | Cₘₐₓ (h) | 175 | 98.8 | 513 |
| administration^{a2} | AUC₀₋ₗₐₛₜ | 2608 | 725 | 4069 |
| (10 mg/kg) | (h*ng/mL) | | | |
| | AUC_{0-inf} | 2975 | 929 | 4085 |
| | (h*ng/mL) | | | |

| way of administration | | compound 4^{b} | compound 5^{b} |
|---|---|---|---|
| intravenous^{a1} | Cl | 35.6 | 51.6 |
| (5 mg/kg) | (mL/min/kg) | | |
| | AUC₀₋ₗₐₛₜ | 1987 | 1603 |
| | (h*ng/mL) | | |
| | AUC_{0-inf} | 2348 | 1617 |
| | (h*ng/mL) | | |
| oral | Cₘₐₓ (h) | 198 | 172 |
| administration^{a2} | AUC₀₋ₗₐₛₜ | 3260 | 1974 |
| (10 mg/kg) | (h*ng/mL) | | |
| | AUC_{0-inf} | 3973 | 1998 |
| | (h*ng/mL) | | |

^{b}male CD-1 mice, mean value of blood samples analyzed separately
^{a1}Intravenous drug delivery solvents is 5% DMSO + 10% Solutol HS-15 + 85% (20% HP-β-CD in water);
^{a2}oral administration drug delivery solvents is 0.1% Tween80 + 0.5% MC in water.

The results showed that the compounds of the present disclosure possessed significant advantages in PK parameters relative to the comparative compound, with a significant increase in the exposure level.

Although specific embodiments of the present disclosure have been described above, it will be appreciated by those skilled in the art that these embodiments are merely illustrative and that many changes or modifications can be made to these embodiments without departing from the principles and spirit of the present disclosure. The scope of protection of the present disclosure is therefore defined by the appended claims.

1. A thiadiazole-substituted compound represented by formula I, a pharmaceutically acceptable salt thereof, a stereoisomer thereof, an isomer thereof or an isotopic compound thereof: 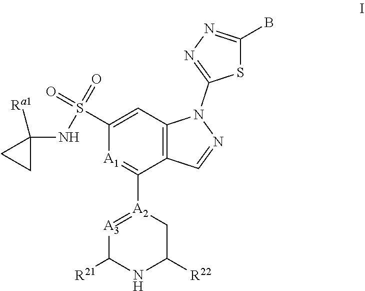
wherein, “ [symbol] ” represents a single bond or a double bond;
B is CF₃ or CHF₂;
A₁ is N or CR^{b1}, R^{b} is hydrogen or halogen; and when B is CHF₂, A₁ is CR^{b1}, R^{b1} is halogen;
A₂ is N, C or CR^{c1}; R^{c1} is hydrogen, halogen, C₁₋₆ alkyl or hydroxyl;
A₃ is CR²³ or CHR²³;
R^{a1} is cyano or C₁₋₆ alkyl;
R²¹ and R²² are independently hydrogen or C₁₋₆ alkyl, and when R²¹ is hydrogen, R²² is C₁₋₆ alkyl;
R²³ is hydrogen, halogen, hydroxyl, cyano, C₁₋₆ alkyl, C₁₋₆ alkyl substituted with one or more R²³⁻¹, C₂₋₆ alkenyl, C₂₋₆ alkynyl, —OC₁₋₆ alkyl, —SC₁₋₆ alkyl, —C(═O)R^{2a}, —NR^{2b1}R^{2b2}, —C(═O)OR^{2c}, —C(═O)NR^{2d1}R^{2d2}, C₃₋₁₀ cycloalkyl, “4- to 12-membered heterocycloalkyl containing 1 to 3 heteroatoms independently selected from O, S and N”, C₆₋₂₀ aryl, “5- to 12-membered heteroaryl containing 1 to 4 heteroatoms independently selected from O, S and N”, C₄₋₈ cycloalkenyl, or, “4- to 8-membered heterocycloalkenyl containing 1 to 3 heteroatoms independently selected from O, S and N”;
R²³⁻¹ is independently halogen, cyano, C₁₋₆ alkyl-O—, hydroxyl, —C(═O)R^{23a}, —NR^{23b1}R^{23b2}, —C(═O)OR^{23e} or —C(═O)NR^{23d1}R^{23d2};
R^{2a}, R^{2b1}, R^{2b2}, R^{2c}, R^{2d1}, R^{2d2}, R^{23a}, R^{23b1}, R^{23b2}, R^{23c}, R^{23d1} and R^{23d2} are independently hydrogen, C₁₋₆ alkyl, or, C₁₋₆ alkyl substituted with one or more halogen;
or, R^{2b1} and R^{2b2}, R^{2d1} and R^{2d2}, R^{23b1} and R^{23b2}, R^{23d1} and R^{23d2} independently together with the N to which they are attached form “3- to 8-membered heterocycle containing 1 to 3 heteroatoms, wherein, one heteroatom is N, other heteroatoms, if any, independently selected from O, S and N”, or “3- to 8-membered heterocycle containing 1 to 3 heteroatoms, wherein, one heteroatom is N, other heteroatoms, if any, independently selected from O, S and N” substituted with one or more R¹⁻²⁻¹;
R¹⁻²⁻¹ is independently independently halogen, C₁₋₆ alkyl, or, C₁₋₆ alkyl substituted with one or more halogen.

2. The thiadiazole-substituted compound represented by formula I, a pharmaceutically acceptable salt thereof, a stereoisomer thereof, an isomer thereof or an isotopic compound thereof according to claim 1, wherein,
“ [symbol] ” represents a single bond or a double bond;
B is CF₃ or CHF₂;
A₁ is N or CR^{b1}, R^{b1} is hydrogen or halogen, and when B is CHF₂, A₁ is CR^{b1}, R^{b1} is halogen;
A₂ is N or C;
A₃ is CR²³ or CHR²³;
R²³ is hydrogen;
R^{a1} is cyano or C₁₋₆ alkyl;
R²¹ and R²² are independently hydrogen or C₁₋₆ alkyl, and when R²¹ is hydrogen, R²² is C₁₋₆ alkyl.

3. The thiadiazole-substituted compound represented by formula I, a pharmaceutically acceptable salt thereof, a stereoisomer thereof, an isomer thereof or an isotopic compound thereof according to claim 1, wherein,
A₂ is N or C;
and/or, A₃ is CR²³ or CHR²³; R²³ is hydrogen;
and/or, when R²¹ and R²² are independently C₁₋₆ alkyl, 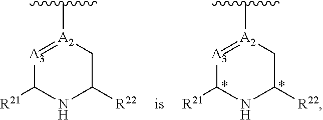
wherein, 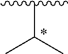 [SMILES: *C(C)C]
represents R conformation, S conformation or a mixture of R and S conformation;
and/or, when the definition of R^{b1}, R^{c1}, R²³, R²³⁻¹, R^{2a}, R^{2b1}, R^{2b2}, R^{2c}, R^{2d1}, R^{2d2}, R^{23a}, R^{23b1}, R^{23b2}, R^{23c}, R^{23d1}, R^{23d2} and R¹⁻²⁻¹ refers to halogen, the halogen is fluorine, chlorine, bromine or iodine;
and/or, when the definition of R^{c1}, R^{a1}, R²¹, R²², R²³, R^{2a}, R^{2b1}, R^{2b2}, R^{2c}, R^{2d1}, R^{2d2}, R^{23a}, R^{23b1}, R^{23b2}, R^{23c}, R^{23d1}, R^{23d2} and R¹⁻²⁻¹ refers to C₁₋₆ alkyl, the C₁₋₆ alkyl is C₁₋₄ alkyl.

4. The thiadiazole-substituted compound represented by formula I, a pharmaceutically acceptable salt thereof, a stereoisomer thereof, an isomer thereof or an isotopic compound thereof according to claim 1, wherein,
A₁ is CF or CH;
and/or, “ [symbol] ” is a single bond, A₂ is N, A₃ is CH₂, R²¹ and R²² are independently C₁₋₆ alkyl;
and/or, R^{a1} is cyano or methyl;
and/or, R²¹ and R²² are independently hydrogen or methyl, and when R²¹ is hydrogen, R²² is methyl.

5. The thiadiazole-substituted compound represented by formula I, a pharmaceutically acceptable salt thereof, a stereoisomer thereof, an isomer thereof or an isotopic compound thereof according to claim 1, wherein, the thiadiazole-substituted compound represented by formula I, having the structure represented by formula II: 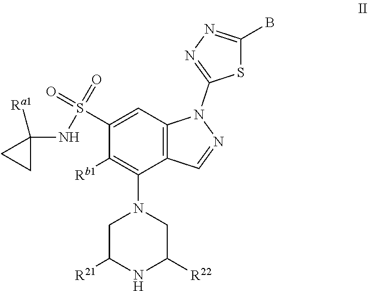
wherein, B is CF₃ or CHF₂;
R^{b1} is hydrogen or halogen, and when B is CHF₂, R^{b1} is halogen;
R^{a1} is cyano or C₁₋₆ alkyl;
R²¹ and R²² are independently C₁₋₆ alkyl.

6. The thiadiazole-substituted compound represented by formula I, a pharmaceutically acceptable salt thereof, a stereoisomer thereof, an isomer thereof or an isotopic compound thereof according to claim 1, wherein,

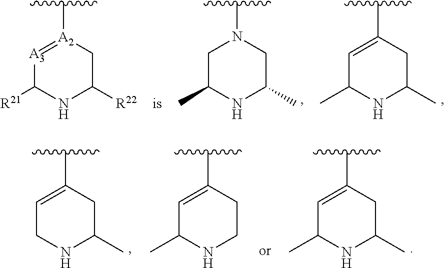

7. The thiadiazole-substituted compound represented by formula I, a pharmaceutically acceptable salt thereof, a stereoisomer thereof, an isomer thereof or an isotopic compound thereof according to claim 1, wherein, the thiadiazole-substituted compound represented by formula I is any one of the following structures:

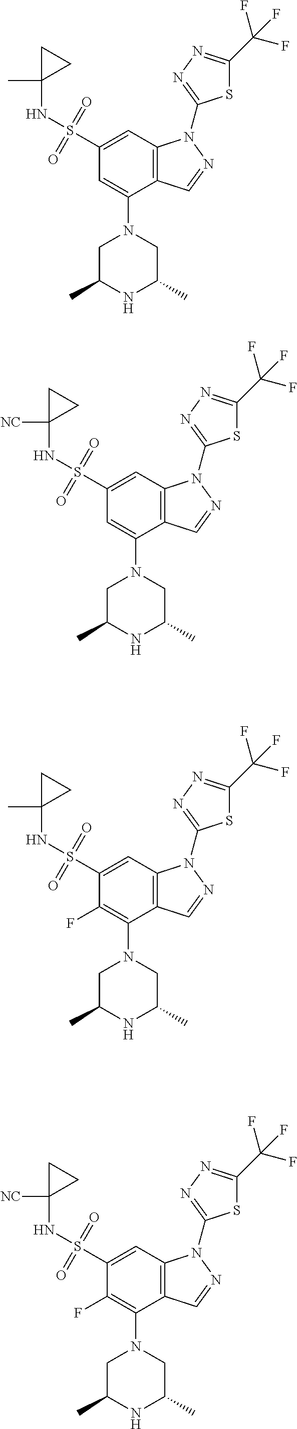 [SMILES: C[C@H]1CN(c2cc(S(=O)(=O)NC3(C)CC3)cc3c2cnn3-c2nnc(C(F)(F)F)s2)C[C@H](C)N1 ; C[C@H]1CN(c2cc(S(=O)(=O)NC3(C#N)CC3)cc3c2cnn3-c2nnc(C(F)(F)F)s2)C[C@H](C)N1 ; C[C@H]1CN(c2c(F)c(S(=O)(=O)NC3(C)CC3)cc3c2cnn3-c2nnc(C(F)(F)F)s2)C[C@H](C)N1 ; C[C@H]1CN(c2c(F)c(S(=O)(=O)NC3(C#N)CC3)cc3c2cnn3-c2nnc(C(F)(F)F)s2)C[C@H](C)N1]

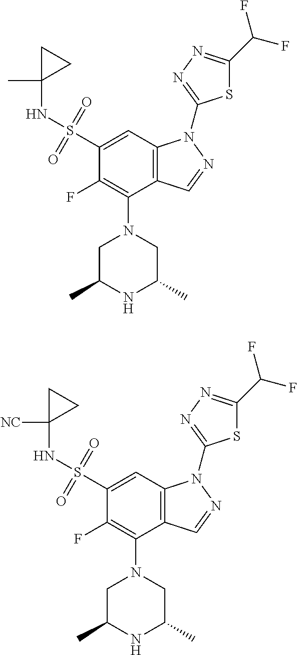 [SMILES: C[C@H]1CN(c2c(F)c(S(=O)(=O)NC3(C)CC3)cc3c2cnn3-c2nnc(C(F)F)s2)C[C@H](C)N1 ; C[C@H]1CN(c2c(F)c(S(=O)(=O)NC3(C#N)CC3)cc3c2cnn3-c2nnc(C(F)F)s2)C[C@H](C)N1]

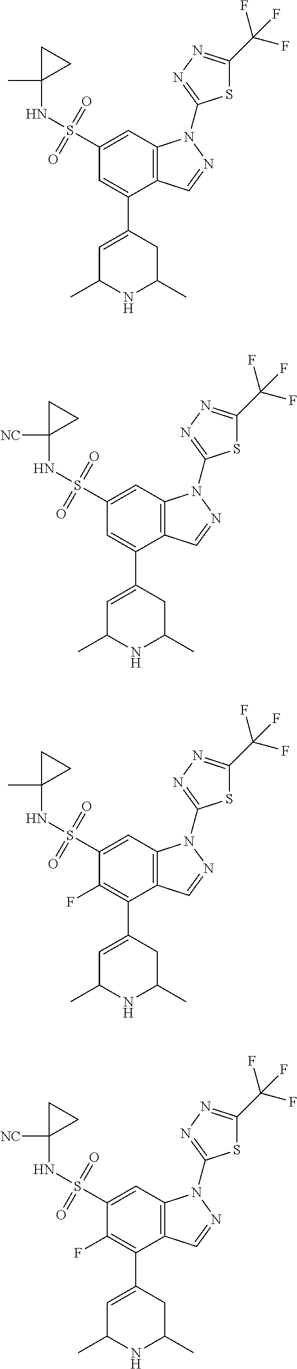 [SMILES: CC1C=C(c2cc(S(=O)(=O)NC3(C)CC3)cc3c2cnn3-c2nnc(C(F)(F)F)s2)CC(C)N1 ; CC1C=C(c2cc(S(=O)(=O)NC3(C#N)CC3)cc3c2cnn3-c2nnc(C(F)(F)F)s2)CC(C)N1 ; CC1C=C(c2c(F)c(S(=O)(=O)NC3(C)CC3)cc3c2cnn3-c2nnc(C(F)(F)F)s2)CC(C)N1 ; CC1C=C(c2c(F)c(S(=O)(=O)NC3(C#N)CC3)cc3c2cnn3-c2nnc(C(F)(F)F)s2)CC(C)N1]

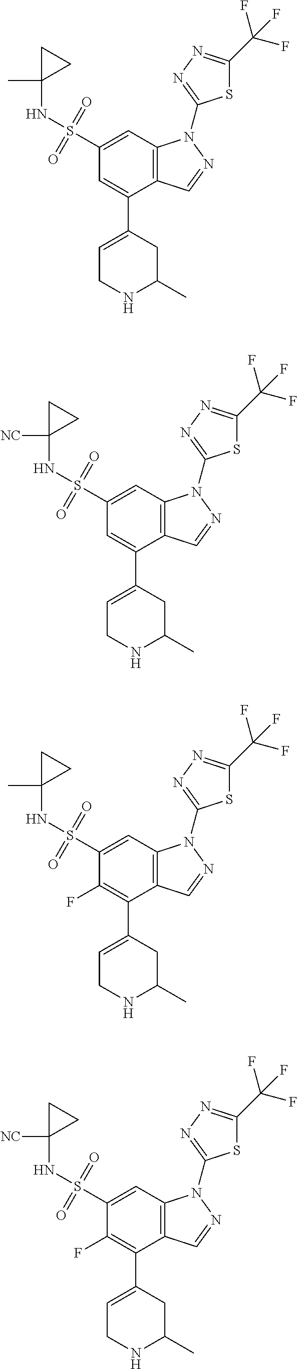 [SMILES: CC1CC(c2cc(S(=O)(=O)NC3(C)CC3)cc3c2cnn3-c2nnc(C(F)(F)F)s2)=CCN1 ; CC1CC(c2cc(S(=O)(=O)NC3(C#N)CC3)cc3c2cnn3-c2nnc(C(F)(F)F)s2)=CCN1 ; CC1CC(c2c(F)c(S(=O)(=O)NC3(C)CC3)cc3c2cnn3-c2nnc(C(F)(F)F)s2)=CCN1 ; CC1CC(c2c(F)c(S(=O)(=O)NC3(C#N)CC3)cc3c2cnn3-c2nnc(C(F)(F)F)s2)=CCN1]

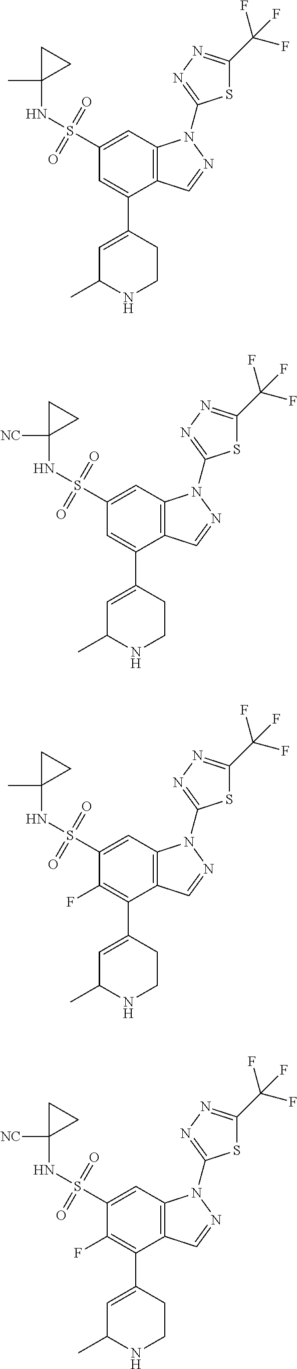 [SMILES: CC1C=C(c2cc(S(=O)(=O)NC3(C)CC3)cc3c2cnn3-c2nnc(C(F)(F)F)s2)CCN1 ; CC1C=C(c2cc(S(=O)(=O)NC3(C#N)CC3)cc3c2cnn3-c2nnc(C(F)(F)F)s2)CCN1 ; CC1C=C(c2c(F)c(S(=O)(=O)NC3(C)CC3)cc3c2cnn3-c2nnc(C(F)(F)F)s2)CCN1 ; CC1C=C(c2c(F)c(S(=O)(=O)NC3(C#N)CC3)cc3c2cnn3-c2nnc(C(F)(F)F)s2)CCN1]

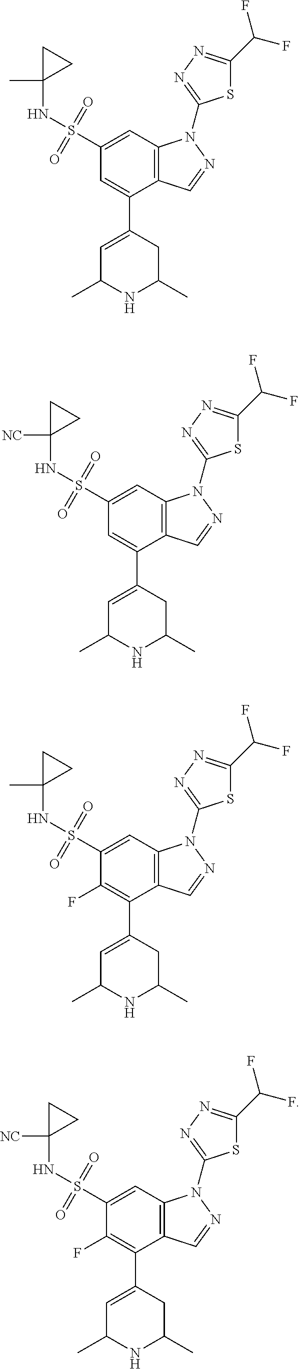 [SMILES: CC1C=C(c2cc(S(=O)(=O)NC3(C)CC3)cc3c2cnn3-c2nnc(C(F)F)s2)CC(C)N1 ; CC1C=C(c2cc(S(=O)(=O)NC3(C#N)CC3)cc3c2cnn3-c2nnc(C(F)F)s2)CC(C)N1 ; CC1C=C(c2c(F)c(S(=O)(=O)NC3(C)CC3)cc3c2cnn3-c2nnc(C(F)F)s2)CC(C)N1 ; CC1C=C(c2c(F)c(S(=O)(=O)NC3(C#N)CC3)cc3c2cnn3-c2nnc(C(F)F)s2)CC(C)N1]

8. A pharmaceutical composition comprising a substance A and a pharmaceutically acceptable excipient, wherein the substance A is a therapeutically effective amount of the thiadiazole-substituted compound represented by formula I, the pharmaceutically acceptable salt thereof, the stereoisomer thereof, the tautomer thereof or the isotopically labeled compound thereof according to claim 1.

9. A method of inhibiting PARG in a subject in need thereof, comprising: administering a therapeutically effective amount of a substance A to the subject, wherein the substance A is the thiadiazole-substituted compound represented by formula I, the pharmaceutically acceptable salt thereof, the stereoisomer thereof, the tautomer thereof or the isotopically labeled compound thereof according to claim 1.

10. A method of treating or preventing a PARG related disease in a subject in need thereof, comprising: administering an effective amount of a substance A, wherein the substance A is the thiadiazole-substituted compound represented by formula I, the pharmaceutically acceptable salt thereof, the stereoisomer thereof, the tautomer thereof or the isotopically labeled compound thereof according to claim 1.

11. In the method for treating or preventing an PARG related disease in a subject in need thereof according to claim 10, wherein the PARG related disease is cancer, the cancer is selected from the group consisting of colon cancer, appendicle cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.
